(19) United States
(12) Patent Application Publication
(10) Pub. No.: US 2026/0240486 A1
(43) Pub. Date: Aug. 20, 2026

(54) CORE COGNITIVE FUNCTION ASSESSMENT

(71) Applicant: VoxNeuro Inc., Hamilton, Ontario (CA)

(72) Inventors: John F. Connolly, Toronto (CA); Kyle Ruiter, Ellershouse (CA)

(73) Assignee: VoxNeuro Inc., Hamilton, Ontario (CA)

(21) Appl. No.: 19/489,535

(22) PCT Filed: Jun. 1, 2024
(86) PCT No.: PCT/IB2024/055366

§ 371 Date: Dec. 2, 2025

Related U.S. Application Data
(60) Provisional application No. 63/470,715, filed on Jun. 2, 2023.

(51) Int. Cl. A61B 5/00; A61B 5/378; A61B 5/38

(52) U.S. Cl. CPC A61B 5/4088; A61B 5/378; A61B 5/38

(57) ABSTRACT

Methods and systems for assessing cognitive function using tracked electrical activity of the brain of the individuals in response to a specific sequence of stimuli in generating data sets, that one or more cognitive functions of a patient can be assessed based on EEG responses obtained during a cognitive health assessment procedure that includes one or more tests. Such cognitive tests and associated functions include: awake and drowsy, auditory evoked potentials, visual evoked potentials, attention and concentration and information processing, and memory. The cognitive functions assessments can be based on at least one ERP response type, including any of: N100, P200 and P300 responses or any combination thereof, and comparison to demographic values. Each of the cognitive functions can be scored and compared to repeated assessments over time.

### CROSS REFERENCE TO RELATED APPLICATIONS

This application claims priority to U.S. Provisional Application No. 63/470,715, filed Jun. 2, 2023, the disclosure of which is herein incorporated by reference in its entirety for all purposes.

### FIELD OF THE INVENTION

Embodiments described herein relate to methods, systems and computer-readable media for assessing cognitive function.

### BACKGROUND

Cognitive health assessments (CHAs) are useful in rehabilitation, diagnostics, prognostics, and assessing various neurocognitive issues. Cognitive health assessments are difficult as cognitive function is challenging to measure. The most relevant metric for assessing and diagnosing the consequences of a condition (e.g. concussion, acquired brain injury, Alzheimer's, etc.) is cognitive function—how a brain responds to stimuli. Increased challenges arise in relation to individuals who appear to be unresponsive (e.g., those who have severe brain damage) and are unable to provide meaningful responses to stimuli. Conventional approaches of performing cognitive health assessments are described in U.S. Pat. No. 6,993,381 entitled “Linking Neurophysiological and Neuropsychological Measures for Cognitive Function Assessment in a Patient” issued on Jan. 31, 2006, and U.S. Pat. No. 11,278,230, entitled “Systems and Methods for Cognitive Health Assessment,” issued on Mar. 22, 2022, each of which is in incorporated herein by reference in its entirety for all purposes.

Conventional approaches tend to be complex and time-consuming and often require repeat visits and for this reason are often performed outside of the clinical settings, where it would be impractical to perform such assessments. Additionally, receiving the result of the cognitive assessments may require additional time as conventional assessments often rely heavily on clinician interpretation. One additional challenge is that a practitioner often unintentionally skews readings on subjective examinations, such as White Coat Syndrome. Even when objective measures of brain activity, such as electroencephalogram (EEG) signals are used in assessments, the clinician's subjective input as to parameters of stimulation and selection of protocols or interpretation of results can result in inaccurate, inconsistent determinations of brain activity or assessment of cognitive function. Therefore, there exists a need for improved methods and systems that rely more on objective measures and analysis of brain function measurements, and less on subjective input from the clinician. Further, there exists a need for more comprehensive assessments that can be more performed more rapidly, consistently and reliably than currently available methods.

### BRIEF SUMMARY

Improved approaches are described herein in relation to assessing cognitive function to determine brain health, and/or diagnosis of a brain condition, and particularly improvements that allow more rapid and reliable cognitive assessments. Applications include mental competency assessments, elderly competency assessments, neurodevelopmental disorder competency assessments, measuring drug effects on brain function, brain injury assessments, and general brain health tracking. Some approaches described herein are especially helpful for seemingly unresponsive individuals as indicators may establish that such individuals may potentially have cognitive function that was not recognized otherwise.

An improved system for assessing cognitive function is described that uses tracked electrical activity of the brain of the individuals in response to a specific sequence of stimuli in generating data sets, which, for example, can be encapsulated as a data structure. The data sets can include EEG signals that track specific response types, at different times (e.g. latencies) and different amplitudes, including, but not limited to, event-related potential (ERP) signal components. Brainwave features including ERPs are tracked in relation to both pre-attentive brain responses and consciously controlled attention responses. The approach uses tracked electrical activity of the brain of the individuals (e.g., during rest or in response to a specific sequence of stimuli) in generating data sets, which, for example, can be encapsulated as a data structure obtained from a sensory device coupled (e.g., attached) to the head of an individual. The data sets can include tracked specific response types, such as ERPs (e.g. N100, P200, and P300), at different times and amplitudes, tracking signal strengths and latencies.

Current approaches of assessment for cognitive health rely heavily on subjective methods of assessment. Reliable, objective data to make an accurate diagnosis and target customized rehabilitation is required and not yet integrated as standard medical practice. The current standard of assessments for concussion and acquired brain injuries (ABIs) is one or a combination of behavioural or neuropsychological tests, CTs or MRI scans. This gold standard is missing the most relevant metric for assessing concussion and ABIs—objective measurements of brain function. Clinicians today are forced to make decisions on patient treatment based on incomplete and often irrelevant information for the condition which means patients are not getting the right treatment fast enough, if at all. Currently, patients have access to either neurocognitive/neuropsychological assessments, or EEG based cognitive assessments; but not both. Accordingly, in one aspect, the methods described herein bridge the gap between these two types of assessments by collecting data values from EEG recordings during neuropsychological assessments. This enables clinicians to assess patients' cognitive function at the biological level. Today, there is no available product to truly, objectively measure cognitive function at both the behavioral and biological levels from a single assessment. Accordingly, in clinic today there is a lack of valuable, unbiased, data on cognitive function available to clinicians and patients.

The technical solutions described herein allow for an assessment that uses neurophysiological, EEG/ERP specific output values to provide cognitive scores indicating the health of a patient's cognitive function. In a specific embodiment, the assessment can employ an Auditory Oddball Test (AOT) and Continuous Visual Memory Test (CVMT) administered on a computer screen while a EEG system records the patient's raw EEG data. The raw EEG data is then parsed by specialized software, such as Cognitive Health Assessment Management Platform (CHAMP) by VoxNeuro, to collect the specific neurophysiological responses elicited during the cognitive test battery. These responses (ERPs), are direct reflections of cognitive function at the neurophysiological level. Obtaining and analyzing multiple ERPs concurrently or in rapid succession and comparison to normative healthy values from healthy controls allows for determination of multiple cognitive functions to better and more rapidly ascertain a patient's cognitive health. Advantageously, such a procedure can be performed rapidly (e.g., one or hour or less). In some embodiments, the entire procedure can be performed in-clinic. It is appreciated however that it is not required that the entire procedure be performed in-clinic, and that some or all of the procedure could be performed elsewhere or at different times.

In one aspect, a computing system for cognitive health assessments is provided, the computing device including at least one processor and computer readable memory. The computing system includes a sensor apparatus connected to one or more electrodes coupled to a patient's head, the one or more electrodes recording brainwave data of the patient, and a stimulus presentation mechanism coupled to one or more sensory output devices (in some embodiments, coupled to the computing system or part of the system), the stimulus presentation mechanism generating a series of programmed stimuli to the patient while the sensor apparatus records the brainwave data of the patient as the patient receives the series of programmed stimuli. These electrodes and the stimulus presentation mechanism operate in concert with one another, based on an automated test protocol. Time-coded data sets are extracted, and brainwave data is correlated with event-based timing. The brainwave (i.e., EEG) data may include various responses from a variety of tests, for example, the Auditory Oddball Test, the Continuous Visual Memory Test, the Measure Mismatch Negativity (MMN), which can include any of: P300, N200, N400, P3a, P3b, N1, among others, or any combination thereof (e.g. N200 and P300). Not all embodiments are so limited and the above are provided as illustrative examples. A waveform feature extractor processing engine is provided that is configured to process the brainwave data of the patient to extract one or more waveform features, the one or more waveform features including for example, one or more ERPs (e.g. P300, N200, N400, etc). In some embodiments, any response detected could be used to begin passive rehabilitation while a patient is still in a coma.

A cognitive health assessment controller is configured to record, using the sensor apparatus, a first portion of brainwave data of the patient during a first resting period during which no stimuli are being presented to the patient, control the stimulus presentation mechanism to present a repeated auditory tone (or, in alternate embodiments, visual stimulus or a combination thereof) to the patient; control the stimulus presentation mechanism to present the repeated auditory tone intermixed with deviant tones (e.g., two or more different sets of tones), and control the waveform feature extractor to track differences in the one or more ERP (e.g. P300, N200) responses recorded in the one or more waveform features during the presentation of the repeated auditory tone to the patient and during the presentation of the repeated auditory tone intermixed with the periodic deviant tones.

Deviant tones may include, for example, random words, recognizable sounds, among others. Deviant sounds can include unfamiliar novel sounds (e.g., dog barks, doorbells), non-salient words (e.g., “NSOW”). Visual stimulus may include repeated visual stimuli followed by deviant visual stimuli. A combination thereof may include deviant tones relative to a visual stimulus, or vice versa. Similarly, vibro-tactile stimuli are also possible, by way of mechanical vibrations (e.g., by way of a mechanical instrument coupled to the body of the patient configured such that individuals are able to detect or respond to stimuli using a sense of touch. Vibrations may be sensed through resonant materials, etc.).

In another aspect, the cognitive health assessment controller is further configured to: control the stimuli presentation mechanism to present the repeated auditory tone intermixed with periodic deviant tones, that are distinct from each other, and track differences in the one or more responses (e.g. MMN, P300 and N200) responses recorded in the one or more waveform features during the presentation of the repeated auditory tone presentation intermixed with the deviant tones and during the presentation of the repeated auditory tone intermixed with the deviant tones.

In another aspect, the cognitive health assessment controller is further configured to: control the stimuli presentation mechanism to present one or more auditory phrases each including one or more nonsensical portions to the patient; and control the waveform feature extractor to track the one or more ERP responses recorded in the one or more waveform features during or proximate to the presentation of the one or more incongruous, nonsensical or unexpected portions.

In another aspect, the processor is configured to augment the brainwave data with time-codes contemporaneous or near contemporaneously with presentation of the series of programmed stimuli. In another aspect, the system includes a video recording device adapted to obtain video data of the patient as the patient undergoing the test. In another aspect, the system delivers a sequence of visual stimuli and the patient is instructed to press a button if a stimulus is repeated to track one or more ERP responses. In another aspect, the patient is in an unresponsive state and the sensor apparatus is configured to record the brainwave data of the patient during one or more additional rest periods where the patient is not receiving the programmed stimuli. In another aspect, the brainwave data of the patient during one or more additional rest periods, where the patient is not receiving the programmed stimuli, is combined with the brainwave data of the patient recorded proximate in time to the presentation of the programmed stimuli.

In another aspect, a method for generating data sets representative of potential cognitive activity of a patient is provided, the method including: recording, using a sensor apparatus connected to one or more electrodes coupled to the patient's head, the one or more electrodes recording brainwave (EEG) data of the patient in respect of a brain of the patient, a portion of brainwave data of the patient during a first resting period during which no stimulus is being presented to the patient; controlling a stimulus presentation mechanism to present a repeated stimulus presentation to the patient; tracking, by the processor: the one or more ERP responses recorded in one or more waveform features during or proximate to the presentation of one or more repeated stimulus to track the brain's ability to work through complex processes to enable complex behaviour; differences in the detection ERPs (e.g. N1, P2, N200, N400, MMN, P300, P3a, P3b, N2b responses) recorded in the one or more waveform features during the presentation of the repeated stimulus to the patient and during the presentation of the repeated stimulus intermixed with the deviants; and analyzing the detected ERP to assess a cognitive function and/or a condition of the patient.

### BRIEF DESCRIPTION OF DRAWINGS

Embodiments will now be described, by way of example only, with reference to the attached figures. It is to be expressly understood that the description and drawings are only for the purpose of illustration and as an aid to understanding, and are not intended as a definition of the limits of the claimed embodiments.

FIG. 1 is a system setup for performing a cognitive health assessment, according to some embodiments.

FIG. 2 shows an exemplary overview of the assessment process, in accordance with some embodiments.

FIG. 3 shows an example cognitive health assessment, in accordance with some embodiments.

FIG. 4 shows example waveforms produced by the awake and drowsy test in a cognitive health assessment, in accordance with some embodiments.

FIG. 5 shows example waveforms produced by auditory evoked potentials in a cognitive health assessment, in accordance with some embodiments.

FIG. 6 shows example waveforms produced by visual evoked potentials in a cognitive health assessment, in accordance with some embodiments.

FIG. 7 shows an example cognitive assessment output showing cognitive scores and waveforms produced by the attention and concentration, information processing and memory tests in a cognitive health assessment, in accordance with some embodiments.

FIG. 8 shows an exemplary progress tracker of cognitive scores across repeated cognitive health assessments, in accordance with some embodiments.

FIG. 9 shows example brain waveforms obtained from a cognitive health assessment, in accordance with some embodiments.

FIG. 10 is an exemplary process of a cognitive health assessment, in accordance with some embodiments.

### DETAILED DESCRIPTION

In one aspect, the current systems and methods revolutionize the way electroencephalography (EEG) is used by utilizing systems that are able to assess cognitive function based on cognitive event-related potentials (ERPs) to provide rapid and reliable cognitive health assessment during a procedure. This can be used to provide initial assessments, which can markedly improved treatment outcomes by providing more accurate diagnosis and allow more in-depth follow-up based on these initial assessments, and follow-up assessments. It is known that EEG-measured ERPs, time-locked to an event or stimulus, are biomarkers of cognitive processes. The systems and methods described herein rely on the potential of cognitive ERPs to provide reliable biomarkers of cognitive function. For patients with suspected cognitive impairment, the systems and methods provide rapid and objective biomarkers to inform clinical decision-making. Advantageously, the system and methods herein provide a novel evidence-based EEG protocol for determining a CHA.

In some embodiments, the systems and methods herein provide a CHA in a sufficiently short period of time such that the assessment and output can be obtained. In some embodiments, the CHA can be completed within less than 2 hours, typically less than one hour (e.g. 30-60 minutes). Advantageously, the procedure is non-invasive, as EEG electrodes sit on the patient's scalp and record the brain's electrical activity through a conductive gel, similar to an ultrasound. The assessment guides patients through a series of neuropsychological tests on a computer while the EEG records tens of thousands of data-points along their brain waves. Among those data points are cognitive ERPs. These ERPs are recorded and are instantly analyzed against a normative database to evaluate and score key cognitive functions. The systems and methods herein utilize multiple cognitive ERPs, such as P300 and N200, that serve as validated biomarkers for core cognitive functions. Such core cognitive functions may include but are not limited to: attention and concentration assessed through the auditory oddball task and analysis of P300; information processing assessed with the auditory oddball task and analysis of N200; and working memory assessed using the continuous visual memory test (CVMT) and analysis of P300.

As noted above, currently, patients have access to either neurocognitive/neuropsychological assessments, or EEG based cognitive assessments; but not both. The most valuable measurements are neurophysiological, not behavioural, as behavioral measures can be influenced by factors irrelevant to assessing cognitive health. Accordingly, in clinic today there is a lack of valuable, unbiased, data on cognitive function available to clinicians and patients.

In one aspect, the invention pertains to an assessment that uses neurophysiological, EEG/ERP specific output values to provide cognitive scores indicating the health of a patient's cognitive function. Moreover, these EEG/ERP values can be obtained during a neurocognitive/neuropsychological assessments, thereby allowing for a faster and more accurate CHA. In some embodiments, the entire CHA procedure can be performed in a relatively short period of time (e.g. less than 2 hours, one hour or less) which can better inform clinical decision making, follow-up testing and assessments, or course of treatments. This allows for identification of appropriate course of treatment earlier after an injury, which in the case of brain injuries, can potentially improve the likelihood of positive patient outcomes. Additionally, the methods and system herein still provide rapid and accurate CHA long after an injury, whereas some conventional tests are limited beyond a narrow window post-injury.

In some embodiments, the assessment includes an Auditory Oddball Test (AOT). In some embodiments, the assessment includes a Continuous Visual Memory Test (CVMT). In some embodiments, the assessment includes both AOT and CVMT tests. It is understood the assessment could further include one or more additional tests. In some embodiments, multiple tests are performed in rapid succession. In some embodiments, subsequent tests conducted are selected based on a result from a prior test. In some embodiments, multiple cognitive functions can be determined, at least in part, from responses received from a single test or multiple tests can use overlapping data sets. The tests can be administered on a computer screen while an EEG system records the patient's raw EEG. The raw EEG data is then parsed by specialized software on the system, such as CHAMP, to collect the specific neurophysiological responses elicited during the cognitive test battery. These responses (ERPs), are direct reflections of cognitive function at the neurophysiological level. Traditional cognitive health assessments are limited because they lack the objective rigor of testing used in other areas of medicine. They are typically reliant on behavioral indicators instead of brain-based biomarkers, making them deeply constrained by subjectivity and bias, and thus clouding measures of cognition. In contrast, the methods and systems herein can expose pure cognitive function, independent of symptomatology and behavior, by measuring cognitive event-related potentials (ERPs) that emerge from underlying biology rather than psychometric behavioral measures assessed through observation, screeners, or neuropsychological batteries. Accordingly, the systems and methods herein can provide objective measures that are not influenced by subjective factors of the test taker or test administrator, unlike traditional cognitive health assessments. Factors like a patient's mood, motivation, literacy, or education level do not affect or confound the results of the cognitive tests. In another aspects, the cognitive tests described herein are also repeatable without a habituation effect that is observed with traditional cognitive health assessments.

Advantageously, the CHA methods and system described herein can provide accurate and rapid assessment without a baseline reading to detect cognitive dysfunction. Some current technologies require a healthy baseline reading to detect cognitive dysfunction. In some embodiments, by utilizing data from a normative database of healthy controls, the methods and systems herein can detect cognitive dysfunction with a single, post-incident measurement, without need for a baseline reading.

In another aspect, the CHA described herein can be used at any time point post-incident. Many current tests and screeners have a narrow window for obtaining accurate insights on the cognitive impact of concussion or traumatic brain injury. Analysis of cognitive ERPs as described herein has shown the sensitivity to detect cognitive dysfunction in cases where it has persisted more than 30 years past the index incident.

In yet another aspect, the methods described herein can be used repeatedly to track change over time without a habituation effect that is observed with traditional cognitive health assessments. Accordingly, the CHA methods described herein can repeat measurements over time, with the capability of tracking recovery from an injury or cognitive decline, such as the progression of dementia. This approach can be used with various types of brain (e.g. various differing types of injuries and disease progressions).

In another aspect, these methods can largely provide access to every type of brain. In some embodiments, the system and methods described herein utilize a lead-based electroencephalography (EEG) system, rather than a cap, that is inclusive for all hair types and styles. Advantageously, the lead-based EEG system is also designed to work even with severe head injuries. In some embodiments, validated ERP analysis is powered by a normative database built through an extensive collection of data from patients of different ages, genders, and ethnicities. This normative database allows the methods to assess cognitive function for any person age 12 and up. The methods described herein can be applied to even the most complex neurological cases, including non-communicative patients (e.g., patients with aphasia) that can't be assessed using traditional methods.

In one aspect, the system setup includes both hardware equipment and specialized software for operating the hardware and performing the CHA methods described herein. It is appreciated that some or all of the hardware equipment can be supplied by a third-party. In some embodiments, the hardware equipment includes a standard CPU and accessories, a portable monitor, headphones (e.g. Etymotic ER 3C Insert Earphones or equivalent), and the one or more EEG sensors can utilize flexible headset (Nautilus Pro 8 Flexible) or a lead-based system. The software can be included on a technical computer operably coupled to the monitor and the EEG sensors. In some embodiments, the software includes specialized software (e.g. CHAMP) and can include features and plugins (e.g. Simulink, HDF-5 plug in) to facilitate operation of the hardware components and integration of the system setup components. In some embodiments, the CHAMP software architecture that works with the EEG hardware consists of a load balancer, web-server, web-client, MQTT broker, database, user service, and file storage service. The purpose of these components is to serve a user-facing portal that provides workflow-management for Cognitive Health Assessments. These components can include any of: a web-server, a web-client, a MQTT broker, a database, a user service and a file storage service. The web-server provides a stateless API through which clients and third parties can interact with CHAMP software. The Web-client provides a web-based user interface that exercises the API, allowing users to view and modify data. The MQTT broker provides a websockets-based connection for live data notifications. The database can include a PostgreSQL database providing data and state persistence. The user service (e.g. AWS Cognito) can provide user and role storage and workflows. The file storage service (e.g. AWS S3) providing HIPAA-compliant file storage. It is appreciated that the above-noted system components are exemplary and various other setups and features could be realized.

In another aspect, the system setup could utilize a more consumer friendly electrode headset have relatively few EEG sensing electrodes (e.g. less than 10, six or fewer electrodes). This approach can be built on for specific medical indications, such as concussion and Alzheimer's. In some applications, this approach is well suited for on-site assessments, such as by medical health providers or caretakers at a remote accident site or at a patient's home or care facility. The EEG & neuropsychological tests can vary according to the needs of the condition.

The current standard of assessment for cognitive function and conditions (e.g. concussion and acquired brain injuries (ABIs)) is one or a combination of behavioural or neuropsychological tests, CTs or MRI scans. This gold standard is missing the most relevant metric for assessing concussions and ABIs—objective measurements of brain function. Clinicians are forced to make decisions on patient treatment based on incomplete and often irrelevant information for the condition which means patients are not getting the right treatment fast enough, if at all. Recent developments have included monitoring and analysis of measured electrical signals of brain activity in the form of EEG data. EEG testing available in most clinics and hospitals is capable of capturing ongoing brain-signals and oscillations passively (e.g., alpha and beta brain waves), and is commonly known as “resting state”. Clinicians typically ‘read’ traditional EEG scans free-form. qEEG is an improvement on traditional EEG in the way it analyses a patient's data, statistically comparing their results to healthy controls. The detection of abnormal brain function as a result of a concussion or acquired brain injury using resting state testing methods with traditional EEG or qEEG is only possible within the first 72 hours post-injury. Otherwise, these methods cannot find useful information regarding a concussion or acquired brain injury because the perturbation of resting brain waves return to normal after 72 hours. Cognitive health assessments as described herein use EEG equipment, and the use of these systems expand the range of measurable brain activity by enabling direct measurement of the brain's responses to stimulation and cognitive tasks. The approach described includes specific computer-controlled sequences and controlled measurements thereof. In some embodiments described herein, enhanced EEG equipment is utilized that is capable of expanding the range of measurable brain activity. The system can include stimuli generating/presenting mechanisms which are selectively actuated in accordance with a specific process or sequence of cognitive function measurements through brain electrical impulse sensing apparatuses. Event related potentials (ERP) measures are tracked to assess different levels of conscious processing and presence of signs of a conscious state predictive of subsequent emergence or potential undiagnosed injury.

In one aspect, the invention pertains to an objective cognitive assessment system and corresponding methods and computer-readable media are described herein. This approach delivers more accurate quantitative insights on patients' brain function that inform, accelerate, and track recovery with unprecedented precision. The systems and methods described herein provide a legitimate measurement of brain function to objectively and specifically inform cognitive management or rehabilitation in one or more core functions such as: auditory processing, visual processing, information processing, automatic attention, reactive attention, concentration, memory, language comprehension and executive function.

In some embodiments, the system includes brain sensing devices (e.g., electrodes), stimuli presentment mechanisms (e.g., display screens, vibration motors, speakers, olfactory release devices), and computer instruction sets for controlling measurement and stimuli presentment is described. The stimuli presentment is controlled to include various repeated, transformed and/or deviant versions of stimuli, and automatic early attentional brain mechanisms and/or consciously controlled attention mechanisms are triggered by such repeated, transformed and/or deviant versions of stimuli. The tracked data, in some embodiments, is processed to extract one or more data sets, which can be used for modifying device operational parameters, updating electronic health records, or as inputs for controlling rendering of a graphical user interface, according to various embodiments.

The embodiments described herein tracks a patient's active responses to a range of tasks and stimuli instead of only at a resting state. The system can include stimuli generating/presenting mechanisms which are selectively actuated in accordance with a specific process or sequence of cognitive function measurements through brain electrical impulse sensing apparatuses. Event related potentials (ERP) measures are tracked to assess different levels of conscious processing and presence of signs of a conscious state predictive of subsequent emergence or potential undiagnosed injury.

In some embodiments, stimuli can include regular stimuli, and generated stimuli that is specifically adapted to be dissonant, incongruous, unexpected, surprising, or deviant (e.g., 2400 tones can be selected, for example, with 82% standard tones (at around 50 ms, 1000 Hz, 80 dB), and three types of deviant tones at 6% of the population each). Deviants can include duration deviants (e.g., 125 ms), frequency deviants (e.g., 1200 Hz), and intensity deviants (e.g., 90 dB SPL). This active engagement with stimuli throughout testing, which can be thought of as a stress-test or performance-test for the brain, is how the system is able to provide objective data on specific brain functions that require rehabilitation, instead of generalizing the higher-level brain function of a patient at resting state—to see if their brain function looks normal or injured.

Information on whether a brain is functioning normally or not is valuable in making an initial diagnosis within the first 72 hours post-injury, but does not allow clinicians to objectively inform targeted cognitive management or rehabilitation plans. Cognitive health assessments, as described herein, can be run at various times, whether an injury happened 2 days ago or 20 years ago, to quantify the functional issues and objectively inform a management or treatment path.

Further explaining the novel-ness of the approach disclosed as compared to traditional EEG, qEEG and other newer EEG based cognitive assessment tools, is in the combination of ERPs recorded, to be able to provide a fulsome report on each core area of cognitive function required to inform a complete and customized cognitive treatment plan, unique to each patient. In some embodiments, the report of core cognitive functions can be referred to as “CORE Results” (e.g. “VoxNeuro CORE Results”). In some embodiments, relying on a combination of two or more of ERPs (e.g., N1, P300, N200, N400) can be particularly useful to asses multiple cognitive functions in order to better understand if a patient's brain function is abnormal as compared to healthy controls.

The system is a computer implemented system that provides physical assessment devices, tools, methods, processes, and computer-readable media storing machine readable instructions, which when executed by one or more processors, perform steps of a method. The system is adapted for performing CHAs that include innovative approaches cognitive assessment to provide functional data that is a direct measurement of brain function activity by using EEG and measuring activity including: ERPs, power spectrum, connectivity, coherence, frequency following responses and related stimulus contexts.

The system, in alternate embodiments, is a software-as-a-service platform (SaaS) hosted on physical distributed resources that are accessible through communications networks. In a SaaS implementation, analyses may be conducted despite local unavailability of extensive computing resources. For example, a mobile hospital or a remote hospital or clinic would be able to conduct the tests on physical devices (e.g., a processor, stimulus effectors [e.g., speakers], data recorders, sensors) that are locally present. The information may then be transmitted to a distributed resource or implementation (e.g., a cloud of computing resources) for processing. Outputs from the system indicative of an analysis, or assessment are generated, and potentially provided back to the mobile/remote hospital or clinic. The SaaS platform may be configured to capture the raw bio-signal data and present a decision support interface that a practitioner can readily view and interact with in preparing a diagnosis.

As noted herein, the brain responses measured can include obligatory sensory responses that can be used to assess, through the selective presentment of stimuli, objective evidence of brain function despite a patient not showing signs otherwise. Obligatory responses can be evoked by the selective presentment of stimuli, such as stimuli that deviate from clusters of identical stimuli, and dissonant sounds/images, and nonsensical lexical pairings, and the descriptions herein relate to mechanisms for tracking electrical brain impulses in respect of “pre-attentive cognitive processes”. Accordingly, the signals tracked herein are obtained without requiring a subject's active involvement. In particular, the P300 subtypes and the MMN have a good correlation with coma awakening.

### Details as to Implementation and Use

In one aspect, the design can be used in all available variants of a standard clinical or research EEG recording system capable of interacting with an internal or external computer system to store the data. In some embodiments, the system is configured to run the methods described herein embodied within a software program (e.g. CHAMP, which is configured to perform the various assessments designed by VoxNeuro), such as any of those described herein or within U.S. Pat. No. 11,278,230, entitled “Systems and Methods for Cognitive Health Assessment, issued on Mar. 22, 2022. In other embodiments, the system can be used in other laboratories and with various other assessment software, and is not limited to use with any particular software.

The following describe aspects of conventional testing that can utilize the assessment approach described herein. It is appreciated that these specific examples are exemplars only and that the assessment approach described above could be used in various other assessments as well.

Historically, facilities for patients with cognitive impairment have relied heavily on patients' progress through trial and error rehabilitation methods. With the cognitive health assessment reports, the rehabilitation providers are able to assess a patient's level of consciousness to predict their outcome and identify their “potential outcome potential”, thus assisting families of patients who need to make a decision on proceeding with treatment, and provide direction for those who show potential based on their results.

For unresponsive wakefulness syndrome (UWS) (vegetative state) or coma patients specifically, the cognitive health assessment reports help the patients' families understand, with the objective data, what levels of consciousness the patient has, facilitating a confident decision in their next steps to pursue treatment, or take the patient off of life support. When cognition is identified meaning the patient has rehab potential, the reliable, quantitative data has helped facilitate the patients' families securing funding for their treatment.

A patient in an unresponsive wakefulness syndrome (UWS) (vegetative state) or within a coma may have some conscious awareness but be unable to respond due to sensory or perceptual impairments, aphasia, motor impairments, subclinical seizure activity, pain, fluctuating arousal, fatigue, and a range of other problems. With conventional assessment tools such a patient would receive an inaccurate diagnosis of UWS (VS). This scenario is far from uncommon.

Without the cognitive health assessment reports, the healthcare providers and families need to make decisions for treatment based off of the behavioural assessments mentioned above—resulting in misdiagnosis rates for UWS (VS) that are consistently estimated at about 40% (Andrews et al., 1996; Childs et al., 1993; Schnakers et al., 2009a,b).

In one assessment, cognitive health assessment reports generated by the analyses produced by the systems and procedures confirm if a concussion or ABI is present, the severity of the injury, specific domains of functional (e.g. neurocognitive) deficit incurred by the injury, which aid in determining areas of focus for rehabilitation.

This allows certainty in results for clinicians to provide targeted and timely methods for rehabilitation. By reducing the need for multiple tests to confirm or disprove a concussion or ABI, patients can get the help they need when they need it—as early into their recovery path as possible. The certainty additionally allows clinicians and patients to avoid trial and error in rehabilitation methods, instead focussing solely on the areas of cognitive rehabilitation the patient needs for their unique condition by following the functions identified as below the healthy norm in their cognitive health assessment report.

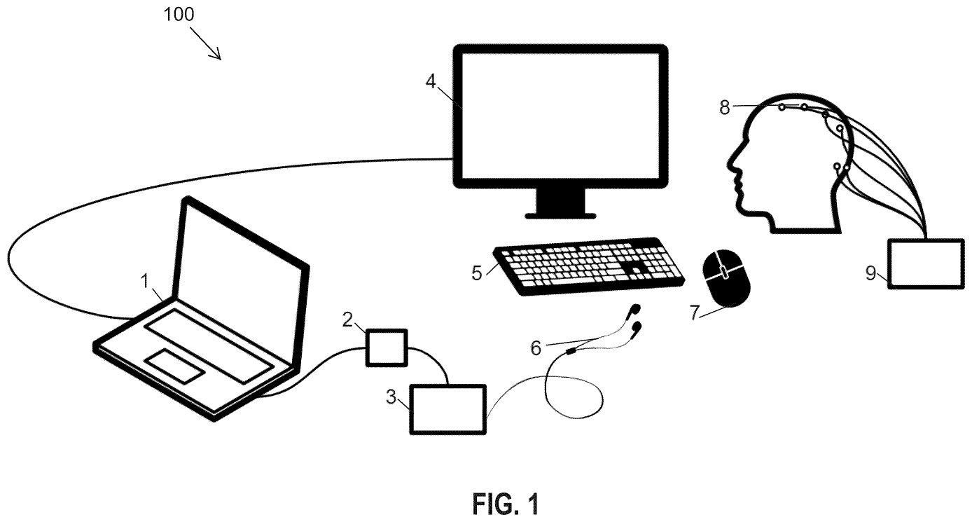

FIG. 1 is an exemplary system setup 100 for conducting a CHA that obtains ERP/EEG data for assessment of cognitive health, in accordance with some embodiments. As shown in FIG. 1, the setup includes a Technician Computer 1 whereby the technician can conduct the procedures described herein, including presenting stimuli to the patient by way of headphones, display controllers, etc. and obtaining/monitoring associated responses. The Technician Computer 1 is communicatively coupled with a memory having programmable instructions recorded thereon that are embodied in software configured to complete the CHA, which can include EEG presentation and acquisition, as described herein, and can further utilize one or more biometric plugins. Acquisition can further include use of specialized software (e.g., VoxNeuro Tests and gRecorder) and a Cognitive Health Assessment Management Platform (CHAMP). Biometric plugins can include an HDF-5 Plug-in. The Technician Computer may be communicatively coupled (e.g. hardwired or wirelessly) to one or more output devices for providing stimulus to the patient. These output devices can include Base Station 2 which is connected to Audiobox 3, which can include an audio speaker output or audio headphones 6 to be worn by the patient for providing audio stimulus. The Technician Computer can further be coupled to a Patient Monitor Display 4, which can be used for any of: completing consent, completing intake forms, communicating information regarding the assessment, and performing the assessment. In this embodiment, the Patient Monitor Display 4 is communicatively coupled with a keyboard 5 and mouse 7, each can be wireless or hardwired. In other embodiments, the patient monitor display can include a touchpad device, such as a tablet. One or more electrodes 8 are placed on the patient (e.g., coupled to the patient's head) to capture brainwaves and function data, including ERPs. The electrodes may be connected to the patient in the form of a cap or other headgear, or individually. In this embodiment, the electrodes are x4 freestanding electrodes placed along the midline using the international 10-20 measurement system, x1 for ground and x1 for reference, typically between 4 and 10 electrodes, preferably 6 total electrodes. It is appreciated other types, number and arrangements of electrodes can be used. The electrodes are connected to Amplifier 9, which receives electrical signals from the electrodes associated with EEG and outputs the EEG data to Base Station 2 connected to the Technician Computer.

During a test session, the system setup facilitates a series of neuropsychological tests for computer presentation, whereby a stimulus presentation program is used to take the patient through a series of steps. The Technician Computer 1 controls administration of the stimulus according to a testing protocol and controls measuring of the brain's electrical activity with the sensing electrodes 8 in response to the stimulus. Technician Computer 1 runs a presentation process to generate programmed stimuli (auditory via Audiobox 3 or visual via Display 4), the patient hears or sees stimulus as their EEG is recorded by sensing electrodes 8, the electrode data is amplified using the EEG equipment and saved by the Technician Computer, optionally overlaid with stimulus markers. These stimulus tests are programmable by the user or an administrator, and the ERP tests/paradigms are programmed to record the ERP responses and correlating one or more cognitive functions at the same time to avoid needing to time-match the two after the fact. Advantageously, the electrical activity signals from the electrodes can be analyzed to determine a given response, such as ERPs, in real time.

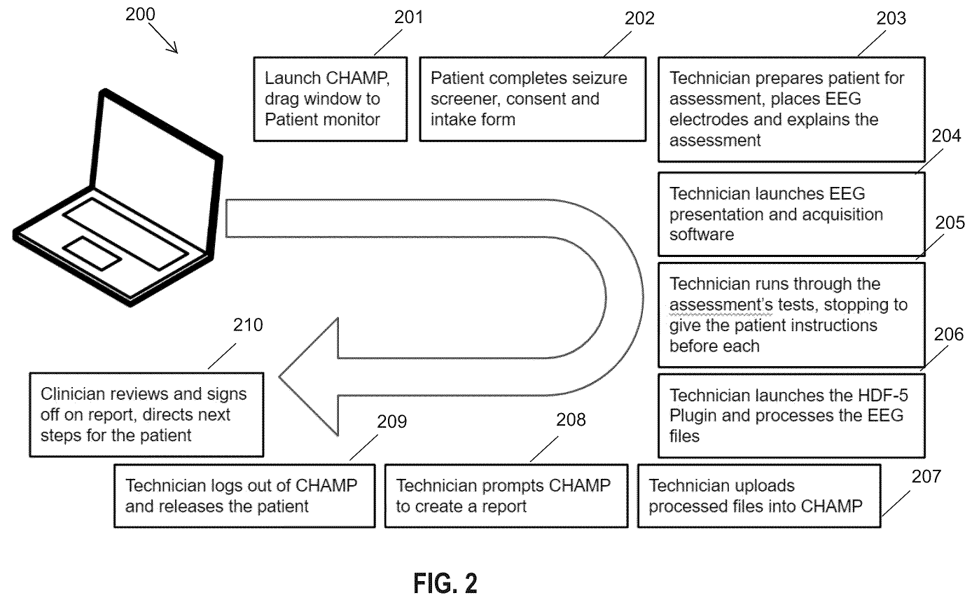

FIG. 2 shows an exemplary overview of the assessment process 200, in accordance with some embodiments. The assessment process 200 includes step 201: launching CHAMP on the Technician; step 202: the patient completes seizure screener, consent and intake form; step 203: technician prepares patient for assessment, places EEG electrodes and explains the assessment to the patient and/or caretaker; step 204, technician launches EEG presentation and response acquisition software; step 205: technician runs through the assessment's test(s), stopping to give the patient instructions before each; step 206: technician launches the biometrics plugin (e.g. HDF-5 Plugin) and processes the EEG files; step 207: technician uploads processed files into CHAMP; step 208: technician prompts CHAMP to create a report; step 209: technician logs out of CHAMP and releases the patient; and step 2010: clinician reviews/approves report and directs next steps for the patient. In some embodiments, next steps entail initiating a course of treatment and/or modification of a course of treatment bases on the reported assessment.

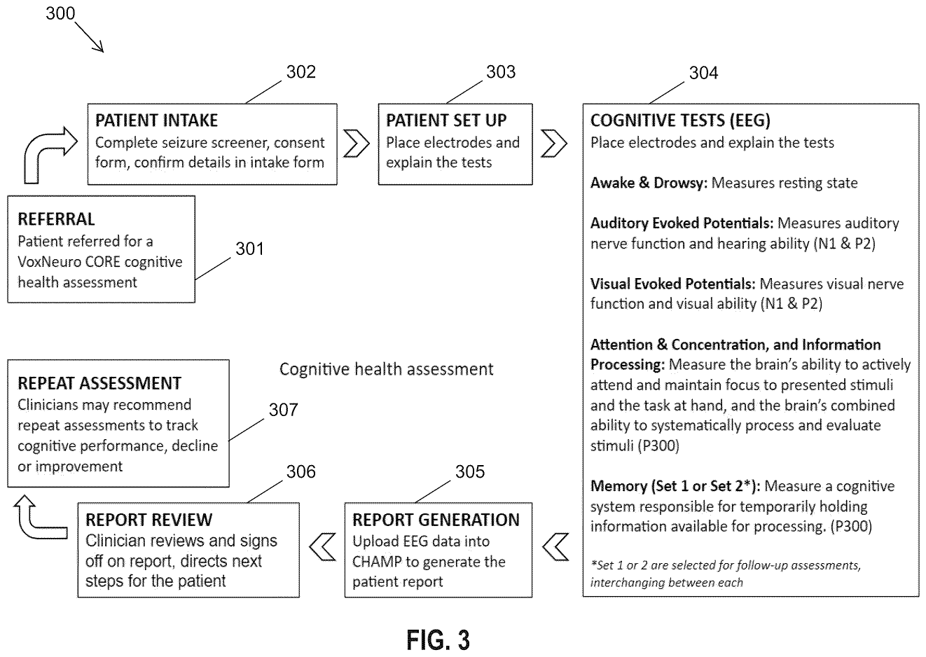

FIG. 3 shows an example cognitive health assessment 300, in accordance with some embodiments. As shown, the cognitive health assessment 300 includes steps of: a patient referral for the CHA 301; the patient intake 302; the patient setup 303 of placing electrodes on the patient's head and explaining the tests; performing one or more cognitive test (EEG) 304; report generation 305; report review 306; and optionally repeat assessments 307. Cognitive tests (EEG) 304 can include but are not limited to: an awake and drowsy test that measures resting state; an auditory evoked potential test that measures auditory nerve function and hearing ability (e.g. N1 and P2); a visual evoked potentials test that measures visual nerve function and visual ability (e.g. N1 and P2 response); an attention and concentration and information processing (ACIP) test that measures the brain's ability to actively attend and maintain focus to presented stimuli, the task at hand and the brain's combined ability to systematically process and evaluate stimuli (e.g. P300), and a memory test (set 1 or set 2) that measures a cognitive system responsible for temporarily holding information available for processing (e.g. P300). It is appreciated that the cognitive test step could include any one or any combination of the tests noted above, or additionally various other tests not listed. Report generation typically includes uploading the EEG data obtained by the patient setup into the CHAMP software to generate the patient report.

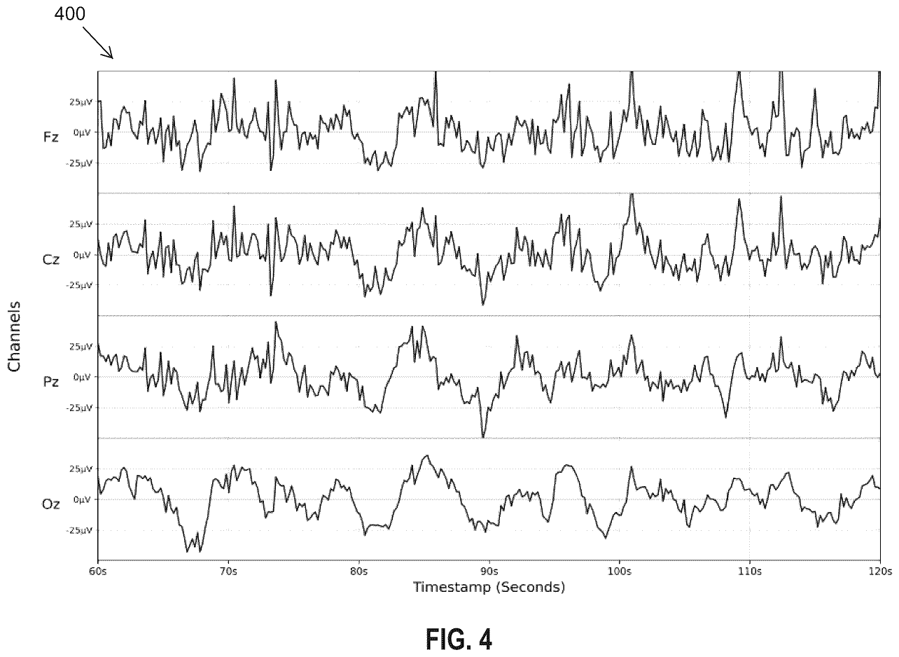

FIG. 4 shows example waveforms 400 produced by the awake and drowsy test in a cognitive health assessment, in accordance with some embodiments. In this embodiment, the waveform displays multiple channels including Fz, Cz, Pz, and Oz, although it appreciated that various other combinations or channels could be utilized in other embodiments. These waveforms are utilizing in determining one or more cognitive functions, typically by comparison to a baseline or demographic averages of responses in healthy individuals.

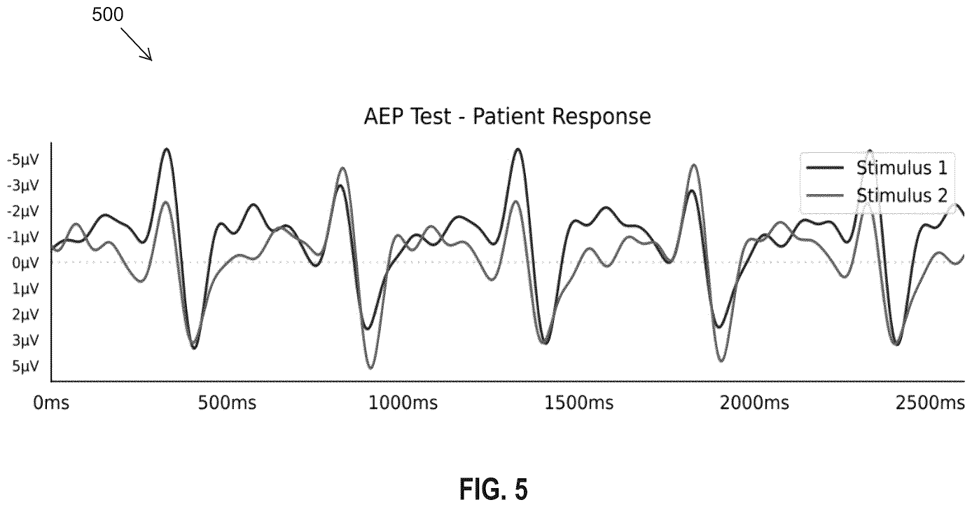

FIG. 5 shows example waveforms 500 produced by auditory evoked potentials (AEP) in a cognitive health assessment, in accordance with some embodiments. In this embodiment, the waveforms of the patient's response to both stimulus 1 and 2 are provided and superimposed. As can be seen, in this case, the stimulus provides similar responses having corresponding peaks. It can be appreciated that an assessment could use only one stimulus.

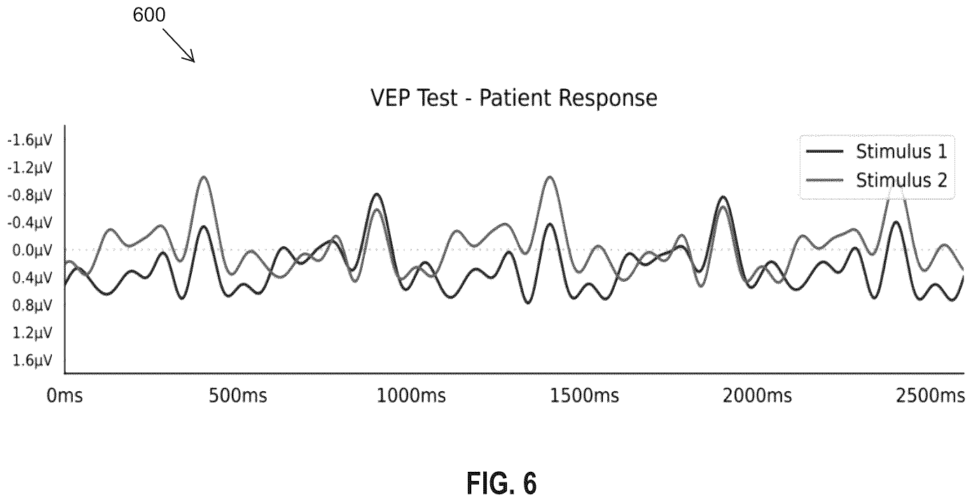

FIG. 6 shows example waveforms 600 produced by visual evoked potentials (VEP) in a cognitive health assessment, in accordance with some embodiments. In this embodiment, the waveforms of the patient's response to both stimulus 1 and 2 are provided and superimposed. As can be seen, in this case, the stimulus provides similar responses having corresponding peaks. It can be appreciated that an assessment could use only one stimulus.

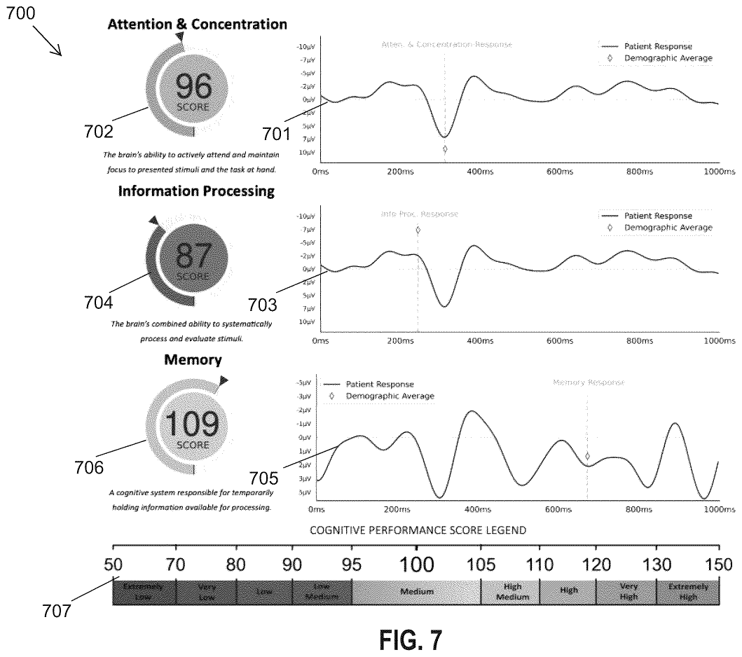

FIG. 7 shows an example cognitive health assessment output 700 showing waveforms produced by the attention and concentration test, information processing test, and memory test in a cognitive health assessment and cognitive scoring for each cognitive function, in accordance with some embodiments. This output provides both a summary details as to the cognitive assessments for each cognitive function being examined. At right, the display shows the waveforms 701, 703, 705 associated with the cognitive functions tested for, namely attention and concentration, information processing and memory. At left, the display shows the associated cognitive scores 702, 704, 706 for each cognitive function based on the demographic average of each function. At bottom, is the cognitive performance score legend 707. This CHA output can be provided on a user interface of the Technician Computer or can be provided as a CHA report output by the system (e.g. printable PDF form).

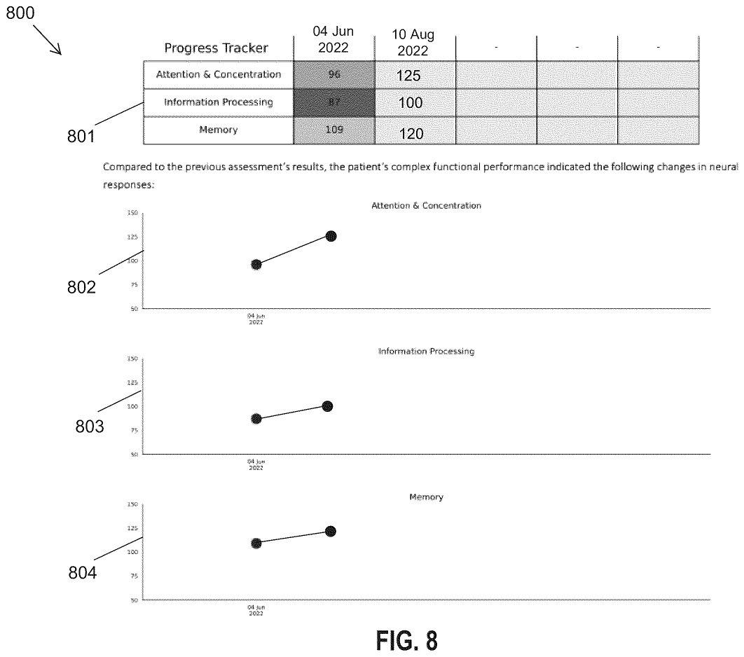

FIG. 8 shows a progress tracker output 800 showing cognitive scores across repeated cognitive health assessments, in accordance with some embodiments. This output can be used to assess changes in cognitive function over time, for example over a period of days, weeks, months or years. In this embodiment, the output shows a single data point obtained from an assessment of Jun. 4, 2022 and Aug. 10, 2022 for each of the cognitive functions: attention and concentration, information processing, and memory. This output shows improvements in each cognitive function over time. As shown, the progress tracker output 800 can include a summary table 801 summarizing the cognitive scores for each cognitive function by date. Additionally, the output can include graphs 802, 803, 804 of cognitive function over time. This output can be provided on a user interface of the Technician Computer or can be provided as pat of the CHA report output by the system (e.g. printable PDF form).

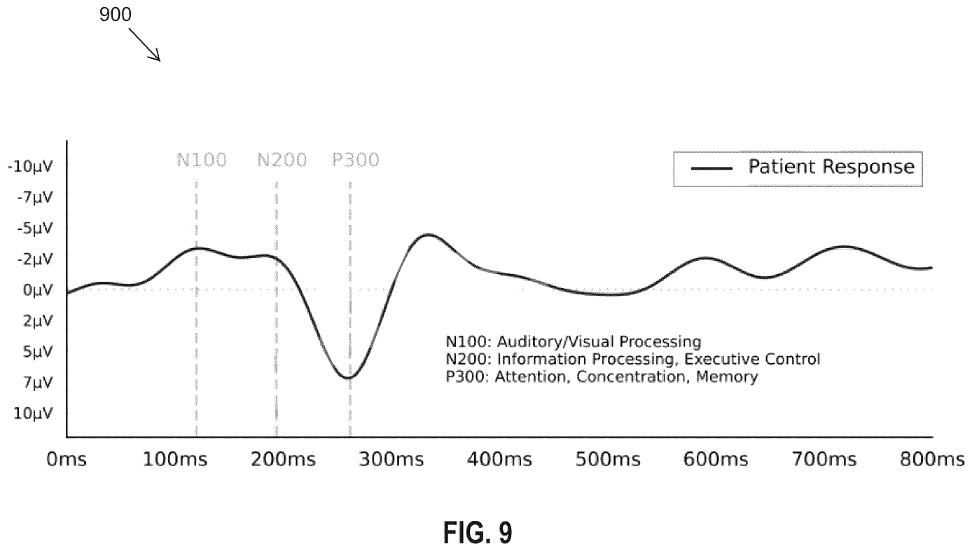

FIG. 9 shows example brain waveforms 900 obtained from a cognitive health assessment, in accordance with some embodiments. Differing brainwave responses are associated with differing cognitive functions. In this embodiment, N100 is associated with auditory/visual processing, N200 is associated with information processing and executive control and P300 is associated with attention, concentration and memory. It is appreciated that these or other responses could be associated with these cognitive functions or other cognitive functions not listed. The relative responses between these differing brainwave responses can be used by the clinician to assess cognitive health in regard to assessing a brain injury or to assess a course of treatment or need for further treatments.

The hardware utilized to capture the brain excitation data can include different types of electrodes, etc. The hardware may need to be adapted based on the size and profile of the individual's head. Example EEG hardware may include g.Tec™, Cadwell™, Biosemi™ system, a BrainProducts™ system or Compumedics Neuroscan™ system, among others.

Paradigm stimulation can be delivered through the stimulation computer and headphones to the patient to stimulate electrical brain activity responsive to the paradigm stimulus. The electrodes capture data values, which are amplified, and provided to an acquisition mechanism and transferred to a data visualization computer. In some embodiments, the stimulation computer is configured to provide stimulus markers, and in some embodiments, receive inputs through a mouse or control pad. The stored data is processed to correlate the data collected by the data visualization computer in concert with the data collected with the stimulation computer such that brain potentials (responses) may be tracked and processed in time-coordination with the presentation of the stimuli.

In some embodiments, the tests/paradigms have been designed with the abilities of the patient in mind (embodiments can include: auditory and vibro-tactile for coma; auditory, vibro-tactile and visual for unresponsive wakefulness syndrome (vegetative state); auditory, visual and vibro-tactile for concussion). Once set up and turned on, the tests are automated. The settings and script have been developed by the Applicants for the ease of analysis by statistical analysts, and the output from a statistical analysis.

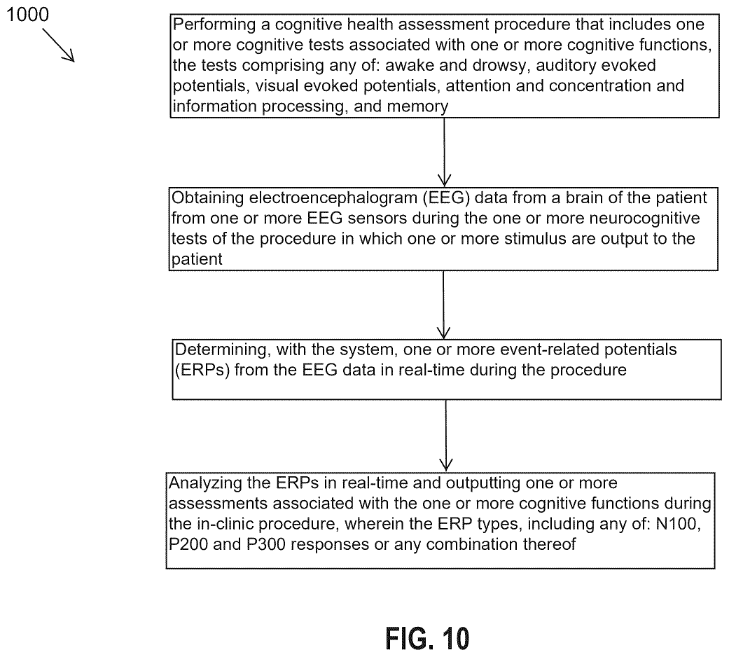

FIG. 10 illustrates an exemplary method to assess cognitive health, which can include assessment of one or more cognitive functions by one or more cognitive tests conducted a procedure, in accordance with some embodiments. The method includes steps of: performing a cognitive health assessment procedure that includes one or more cognitive tests associated with one or more cognitive functions, the tests comprising any of: awake and drowsy, auditory evoked potentials, visual evoked potentials, attention and concentration and information processing, and memory; obtaining EEG data from a brain of the patient from one or more EEG sensors during the one or more neurocognitive tests of the procedure in which one or more stimulus are output to the patient; determining, with the system, one or more ERPs from the EEG data in real-time during the procedure; and analyzing the ERPs in real-time and outputting one or more assessments associated with the one or more cognitive functions during the procedure, wherein the ERP types, including any of: N100, P200 and P300 responses or any combination thereof. While this method is contemplated being performed, it is appreciated that in some embodiments, this or similar procedures could be performed on-site at a remote location, site of injury or at home or a care facility for select patients.

The hardware utilized to capture the brain excitation data can include different types of electrodes, etc. The hardware may need to be adapted based on the size and profile of the individual's head. Example EEG hardware may include g.Tec™, Cadwell™, Biosemi™ system, a BrainProducts™ system or Compumedics Neuroscan™ system, among others.

Paradigm stimulation can be delivered through the stimulation computer and headphones to the patient to stimulate electrical brain activity responsive to the paradigm stimulus. The electrodes capture data values, which are amplified, and provided to an acquisition mechanism and transferred to a data visualization computer. In some embodiments, the stimulation computer is configured to provide stimulus markers, and in some embodiments, receive inputs through a mouse or control pad. The stored data is processed to correlate the data collected by the data visualization computer in concert with the data collected with the stimulation computer such that brain potentials (responses) may be tracked and processed in time-coordination with the presentation of the stimuli.

The definitions and the outcomes of each ERP responses (generated in the Cognitive Function Tests) are most easily clinically relatable to neuropsychologists as the data matches testing that they would otherwise complete offline with manual measurements, which are inherently subjective. The testing validates the outcomes of these tests and captures the responses as purely objective data, extracting any potential for interpretation.

Although the specific ERP responses are outside the existing measures occupational therapists (OTs), physical therapists (PTs) and speech language pathologists (SLPs) utilize (today), reports generated by the system described herein are also extremely clinically valuable to these types of therapeutic interventions in addition to neuropsychology as they: track progress, and with repeated assessments throughout rehabilitation, the reports provide a tool for tracking a patient's progress, and aids in confirming effective methods of treatment.

Functional improvements are measurable approximately 30 days before those improvements translate to behaviour, which provides early encouragement and verification that the chosen methods of rehabilitation are working. The reports also help set recovery expectations, whereby the reports help the clinician, patient and family understand the length of time, and level of involvement required in a rehabilitation plan. The reports also provide benchmarks of the patient's ‘norm’ against age/sex matched controls and their personal benchmarks (when available) to confirm if the patient is ready to return to activities or return to regular activity or play without any uncertainty, for concussed patients specifically. The reports, for example, help a clinician indicate if the patient has a concussion or ABI, and what type it is (e.g., differentiating between coma and/or unresponsive wakefulness syndrome (vegetative state), and if a concussion is present), the severity of that concussion or ABI, the areas of function that are impacted. Reports generated may include, for example: a) Patient reports for specific conscious state: i) coma, ii) unresponsive wakefulness syndrome (vegetative state), and iii) concussion; and b) Reports formatted specifically for clinical rehabilitation and personal injury law firms, among others.

The processor is configured for processing the data sets to identify one or more differences in the N1, P2, N200, N400, MMN, P300, P3a, P3b, N2b responses recorded in the one or more waveform features during the presentation of the repeated auditory tone or visual image presentation to the patient and during the presentation of the repeated auditory tone or visual image intermixed with the deviants, and the one or more P3b responses recorded in one or more waveform features in response to complex visual pattern stimuli, some of which are repeated throughout the sequence.

The processor then generates a data set based on the extracted waveform features. In some embodiments, the data set including data fields corresponding to at least one of an automatic attention metric based at least on the differences in the one or more MMN responses, a reactive attention metric based at least on the differences in the one or more ERP responses (e.g. P300, N200).

In some embodiments, the tool is a standalone, special purpose machine that is adapted for use in a clinical setting. The special purpose machine may have specialized software and hardware, such as optimized integrated circuits or field programmable gate arrays. For example, the tool may be provided on a medical cart, coupled to a patient (e.g., following a concussion or a comatose patient), and the cognitive health assessments are measured as stimuli are presented (e.g., sound tones, vibrations, visual stimuli), or between when stimuli are presented. These stimuli are presented even to patients who are otherwise unresponsive (individuals with locked in syndrome, etc.). The tool includes EEG hardware, computer processors, and software that are specially configured in relation to performing the above tests. Stimuli and acquisition software are adapted based on the techniques described above, including statistical analysis and measurements, which responsive to the measurements, are used in generating decision support interfaces.

Examples of computer storage media include RAM, ROM, EEPROM, flash memory or other memory technology, CD-ROM, digital versatile disks (DVD), blue-ray disks, or other optical storage, magnetic cassettes, magnetic tape, magnetic disk storage or other magnetic storage devices, or media which can be used to store the desired information and which can be accessed by an application, module, or both. Computer storage media may be part of the mobile device, tracking module, object tracking application, etc., or accessible or connectable thereto. Applications herein described are implemented using computer readable/executable instructions that may be stored or otherwise held by such computer readable media.

Thus, alterations, modifications and variations can be affected to the particular embodiments by those of skill in the art without departing from the scope of this disclosure. In further aspects, the disclosure provides systems, devices, methods, and computer programming products, including non-transient machine-readable instruction sets, for use in implementing such methods and enabling the functionality described previously. Although the disclosure has been described and illustrated in exemplary forms with a certain degree of particularity, it is noted that the description and illustrations have been made by way of example only. Numerous changes in the details of construction and combination and arrangement of parts and steps may be made. Except to the extent explicitly stated or inherent within the processes described, including any optional steps or components thereof, no required order, sequence, or combination is intended or implied. As will be understood by those skilled in the relevant arts, with respect to both processes and any systems, devices, etc., described herein, a wide range of variations is possible, and even advantageous, in various circumstances. It is further appreciated that any of the “aspects” of the invention can be considered separately from various other aspects noted above, and that such aspects can be considered to be used independently or in combination with any other aspect described herein.

In this respect, before explaining at least one embodiment in detail, it is to be understood that claimed embodiments is not limited in its application to the details of construction and to the arrangements of the components set forth in the following description or illustrated in the drawings. Other embodiments are possible and carried out in various ways. Also, it is to be understood that the phraseology and terminology employed herein are for the purpose of description and should not be regarded as limiting.

1. A method for assessing cognitive health of a patient, the method comprising:
performing a neurocognitive testing procedure that includes conducting one or more cognitive tests for assessing one or more cognitive functions, the procedure comprising:
obtaining electroencephalogram (EEG) data from a brain of the patient from one or more EEG sensors during the one or more cognitive tests of the procedure in which one or more stimulus are output to the patient;
determining, with the system, one or more event-related potentials (ERPs) from the EEG data in real-time during the procedure; and
analyzing the ERPs in real-time and outputting one or more assessments associated with the one or more cognitive functions.

2. The method of claim 1, wherein neurocognitive testing procedure is completed within one hour or less.

3. The method of claim 1, wherein the one or more cognitive tests comprise an Auditory Oddball Test (AOT) and a Continuous Visual Memory Test (CVMT).

4. The method of claim 1, wherein the ERP data includes P300 and N200 responses.

5. (canceled)

6. The method of claim 1, wherein the one or more cognitive tests and associated functions comprise: attention and concentration, awake and drowsy, auditory evoked potentials, visual evoked potentials, attention and concentration and information processing, and working memory.

7. (canceled)

8. The method of claim 5, wherein the attention and concentration is assessed through the AOT test and analysis of P300; information processing is assessed with the AOT test and analysis of N200; and working memory is assessed using the CVMT and analysis of P300

9. The method of claim 1, wherein outputting one or more assessments associated with the one or more cognitive functions comprises outputting a cognitive score for each of the one or more cognitive functions.

10. The method of claim 9, wherein the cognitive score is based, at least in part, on demographic averages of responses in healthy individuals, optionally the demographic averages of responses are obtained from a normative database accessed by the cognitive health assessment system.

11. (canceled)

12. The method of claim 1, wherein the method is performed any time post injury, such as beyond 72 hours post injury.

13. (canceled)

14. The method of claim 1, wherein the method is performed after a severe head injury, wherein the one or more EEG electrodes are lead electrodes suited for placing on a patient having a severe head injury.

15. (canceled)

16. The method of claim 1, wherein the method is performed in patients that are unresponsive due to sensory or perceptual impairments, aphasia, motor impairments, subclinical seizure activity, pain, fluctuating arousal, or fatigue.

17. The method of claim 1, further comprising repeating the steps in claim 1 at a subsequent time to track recovery or changes over time and optionally outputting a report that graphs multiple cognitive scores over time for each cognitive function assessed over time.

18. (canceled)

19. The method of claim 1, wherein the method is performed to assess any of: a brain injury, mental competency, elderly competency, neurodevelopmental disorder competency, brain damage, drug effects on brain function, and general brain health tracking.

20. A system for assessing cognitive health of a patient, the system comprising:
one or more sensing electrodes for placement on a head of the patient, the one or more electrodes configured for recording EEG data with respect to a brain of the patient;
one or more sensory output devices configured for providing sensory stimuli to the patient;
a technician computing device comprising a processor operably coupled to a memory having programmable instructions recorded thereon that are configured to:
perform a neurocognitive testing procedure that includes conducting one or more cognitive tests for assessing one or more cognitive functions, the procedure comprising:
obtaining electroencephalogram (EEG) data from a brain of the patient from one or more EEG sensors during the one or more cognitive tests of the procedure in which one or more stimulus are output to the patient;
determining, with the system, one or more event-related potentials (ERPs) from the EEG data in real-time during the procedure; and
analyzing the ERPs in real-time and outputting one or more assessments

21. (canceled)

22. The system of claim 20, wherein the programmable instructions are configured to perform multiple cognitive tests including AOT and a CVMT.

23. The system of claim 20, wherein the programmable instructions are configured to perform the cognitive tests that obtain ERP data that includes P300 and N200 responses.

24. The system of claim 20, wherein any of:
the programmable instructions are configured such that each of the one or more cognitive functions are assessed based on at least one ERP response type, including any of: N100, P200 and P300 responses or any combination thereof;
the programmable instructions are configured to perform such that one or more cognitive functions comprise any of: attention and concentration; information processing; working memory, or any combination thereof; and
the programmable instructions are configured to perform such that one or more cognitive tests and associated functions comprise: awake and drowsy, auditory evoked potentials, visual evoked potentials, attention and concentration and information processing, and memory.

25. (canceled)

26. (canceled)

27. The system of claim 24, wherein the attention and concentration is assessed through the AOT by analysis of P300 responses; information processing is assessed with AOT by analysis of N200 responses; working memory is assessed with CVMT by analysis of P300 responses.

28. The system of claim 20, wherein outputting one or more assessments associated with the one or more cognitive functions comprises outputting a cognitive score for each of the one or more cognitive functions, optionally the cognitive score is based, at least in part, on demographic averages of responses in healthy individuals.

29. (canceled)

30. A non-transitory machine readable medium storing a program for performing a cognitive health assessment system comprising: a sensor apparatus connected to one or more electrodes for placement on a head of the patient and configured for recording EEG data with respect to a brain of the patient during a neurophysiological assessment of a cognitive health of a patient based on a behavioral response, one or more sensory output devices configured for providing sensory stimuli to the patient, wherein the program, when executed by one or more processors of the system, causes the one or more processors to perform the method steps of claim 1.
